(19) United States
(12) Patent Application Publication
(10) Pub. No.: US 2010/0239562 A1
(43) Pub. Date: Sep. 23, 2010

(54) Kv CHANNELS IN NEURODEGENERATION AND NEUROPROTECTION

(75) Inventors: Avindra Nath, Ellicott City, MD (US); Tongguang Wang, Lutherville, MD (US); Peter Calabresi, Lutherville, MD (US)

Correspondence Address: BANNER & WITCOFF, LTD., 1100 13th STREET, N.W., SUITE 1200, WASHINGTON, DC, 20005-4051, US

(73) Assignee: THE JOHNS HOPKINS UNIVERSITY, Baltimore, MD (US)

(21) Appl. No.: 12/301,771

(22) PCT Filed: May 22, 2007
(86) PCT No.: PCT/US07/12139

§ 371 Date: Nov. 19, 2009

Related U.S. Application Data
(60) Provisional application No. 60/802,350, filed on May 22, 2006.
(60) Provisional application No. 60/802,824, filed on May 23, 2006.

(51) Int. Cl. A61K 39/395; A61K 38/16; A61K 31/35; A61K 31/352; A61P 25/28; A61P 25/16; A61K 31/7088; C12Q 1/37; G01N 33/53

(52) U.S. Cl. 424/130.1; 514/455; 514/456; 514/44 R; 514/44 A; 435/23; 435/7.21

(57) ABSTRACT

A family of potassium channels (Kv) are expressed in neurons when they are damaged. Blockers of these channels protect neurons from several different types of insults, whether due to disease or trauma. Furthermore, blockers of these channels promote neurite outgrowth in neural progenitor cells. These findings permit methods of treating as well as methods for identifying and developing drugs for neurological diseases where injury to neurons may occur.

This application claims the benefit of provisional applications 60/802,350 filed May 22, 2006 and 60/802,824 filed May 23, 2006, the entire contents of which are expressly incorporated herein.

### TECHNICAL FIELD OF THE INVENTION

This invention is related to the area of drug development and treatment of neurodegenerative diseases. In particular, it relates to neuroprotection and stimulation of neural growth.

### BACKGROUND OF THE INVENTION

Cerebral atrophy and neuronal injury are important correlates of long term disability in patients with chronic inflammatory diseases such as multiple sclerosis (MS). Although the mechanisms of neuronal injury are not well understood, several lines of evidence suggest that inflammatory infiltrates may be key factors in mediating cerebral atrophy and black holes or axonal transaction in patients with MS. For example, neuroimaging studies suggest that ring enhancing patterns on MRI contribute to severe brain atrophy in patients with MS(1). Several studies have shown that patients exhibit significant brain atrophy in the earliest stages of MS and that CNS atrophy and axonal loss may develop at a faster rate in the first few years of disease onset (2-7). Other reports have suggested that the rate of progression of CNS atrophy may be greater in more advanced relapsing—remitting (RR) phases of disease (8-10). Further, treatment with pulse methylprednisolone (9), blockade of cell migration with anti-VLA-4 monoclonal antibody (11) or modulation of T cell phenotype with glatiramer acetate (12) resulted in less black hole formation on T-1 MR images and less loss in cerebral volume. These studies support the role of lymphocytic infiltrates in mediating neuronal damage. Further, pathological studies from MS patients, have also shown that axonal injury may occur early during lesion evolution and is not related to demyelinating activity and thus suggesting that axonal injury is an independent process (13-15). There is also evidence for apoptotic loss of neurons in the cerebral cortex (16). Other neuroinflammatory diseases associated with T cell infiltrates in the brain such as Rasmussen's encephalitis (17) and immune reconstitution syndrome in patients with HIV infection (18) are also associated with neuronal injury but have not been as well studied.

Immune abnormalities in MS include activated T cells in the blood and cerebrospinal fluid. T cells reactive to myelin components are found in a state of heightened activation and differentiation in patients with MS but not controls (19-21). While it is widely hypothesized that autoreactive T-cells recognize myelin proteins and initiate the inflammatory cascade, recent reports also indicated that activated CD8 T cells also induce axonal damage in vivo (22, 23). Further activated T cells can also cause direct neuronal toxicity in vitro through a contact dependent mechanism (24). But whether activated T cells induce neuronal toxicity through released soluble factors is still unclear. MRI studies suggest there is axon damage in normal appearing white matter and histopathological reports document low level neuronal and axonal damage in areas devoid of inflammatory cells, which suggest the possibility that soluble cytotoxic mediators may cause distant damage in MS tissues (23). Further, GrB staining has been seen in MS brain tissue and was recently implicated as being as important mediator of tissue damage in progressive MS (25,26) and in other neuroinflammatory disorders such as Rasmussen's encephalitis (27)

Although the specific mechanism underlying T cell-induced direct neuronal toxicity is unknown, an important mechanism used by cytotoxic T cells to induce target cell death is through the granule exocytosis pathway involving the delivery of lymphocyte granule toxins to target cells (28). Two major constituents of the lymphocyte granules are serine protease granzymes and the Membrane disrupting protein perforin. Among many members of the granzymes, granzyme B (GrB) is the best-characterized because of its strong proapoptotic activities. GrB is a 32-kDa serine protease and cleaves its substrates on the carboxy side of acidic residues, especially aspartate. While perforin made pores in the plasma membrane was originally thought to be necessary for GrB's entry into the target cell, it has been shown that GrB can be taken up by target cells independent of perforin (29). The uptake of GrB into the target cell has been shown to be partly mediated through the mannose-6-phosphate receptor (MPR). However, MPR is not an exclusive mechanism for GrB uptake, as MPR deficient cells remained as vulnerable to GrB similar to wild-type cells (30).

GrB signaling pathways involves activating caspases, a large family of endogenous cytosolic proteases that mediate apoptosis (31). GrB also regulates mitochondrial outer membrane permeabilization via the cytosolic, pro-apoptotic protein Bid (32). It has been shown that GrB cleaved Bid translocates to the mitochondria and interacts with other pro-apoptotic proteins such as Bax and Bak to induce release of cytochrome C from the mitochondria (33). A caspase-independent pathway of GrB-induced apoptosis has also been identified in various cell lines exposed to GrB and perform in the presence of a caspase inhibitor, z-VAD-fink, when cell death could still proceed even though the nuclear apoptotic changes were largely abrogated (34).

Type n potassium channel is the dominant voltage gated K+ channel in human T cells (DeCoursey et al, 1984. Voltage-gated K+ channels in human T lymphocytes: a role in mitogenesis? Nature 307: 465-8.; Matteson and Deutsch, 1984. K channels in T lymphocytes: a patch clamp study using monoclonal antibody adhesion. Nature 307: 468-71.). This channel is encoded by the Kv1.3 gene and therefore referred to as the Kv1.3 channel within the Shaker family of Kv channels. Kv1.3 is a voltage-gated channel assembled from four identical, non covalently linked subunits of about 500 amino acids. Its gating is controlled by the membrane potential. Changes in the membrane potential are detected by a “voltage sensor” region that contains positively charged residues in every third position, the so called “gating charges.” This voltage sensor is located in the S3 and S4 domains of the channel subunits. Changes in membrane potential result in movement of the voltage sensor which is coupled to the gate, thus voltage-sensitive gating is accomplished (Panyi et al, 2004. Ion channels and lymphocyte activation. Immunol Lett 92: 55-66). Kv1.3 has relatively slow kinetics that allow significant K⁺ efflux through activated channels before they enter the non-conducting state. Most of our current understanding of these channels comes from studies in lymphocytes, where they are implicated in activation and proliferation. However, these channels are also abundant in brain and have been localized to hippocampal neurons (Ohno-Shosaku et ai, 1996. Presence of the voltage-gated potassium channels sensitive to charybdotoxin in inhibitory presynaptic terminals of cultured rat hippocampal neurons. Neurosci Lett 207: 195-8). However, their physiological properties or alteration in pathological states have not been studied in the brain.

There is a continuing need in the art to develop treatments for neurological diseases and injuries that will protect existing neurons from destructions and stimulate growth and or development of new neurons and neuronal processes.

### SUMMARY OF THE INVENTION

According to one embodiment of the invention a method is provided for identifying test agents as neuroprotective agents. A first sample of cells which express Kv1.3 is contacted with Granzyme B in the presence of a test agent. A second sample of said cells is contacted with Granzyme B in the absence of a test agent. Viability of the cells in the first and second samples is determined after the contacting. Determined viability of the cells in the first and second samples is compared. The test agent is identified as a candidate neuroprotective agent if the viability of the cells is higher in first sample than in the second sample.

According to another embodiment of the invention a method is provided for treating a mammal with a neurodegenerative disease or neural injury. A specific Kv 1.3 channel inhibitor is administered directly to the central nervous system (CNS) of the mammal. Loss of viable neurons is thereby inhibited, and/or growth of neuronal processes is thereby stimulated, and/or proliferation of neuronal precursor cells is thereby stimulated.

According to another embodiment of the invention a method is provided for treating a mammal with a neural injury or a neurodegenerative disease not associated with pathological T cell activation. A specific Kv 1.3 channel inhibitor is administered to the mammal. Loss of viable neurons is thereby inhibited, and/or growth of neuronal processes is thereby stimulated, and/or proliferation of neuronal precursor cells is thereby stimulated.

According to another embodiment of the invention a method is provided for treating a mammal with a neurodegenerative disease or neural injury. A specific inhibitor of Kv 1.3 channel expression is administered directly to the central nervous system (CNS) of the mammal. Loss of viable neurons is thereby inhibited, and/or growth of neuronal processes is thereby stimulated, and/or proliferation of neuronal precursor cells is thereby stimulated.

According to another embodiment of the invention a method is provided for treating a mammal with a neural injury or a neurodegenerative disease not associated with pathological T cell activation. A specific inhibitor of Kv 1.3 channel expression is administered to the mammal. Loss of viable neurons is thereby inhibited, and/or growth of neuronal processes is thereby stimulated, and/or proliferation of neuronal precursor cells is thereby stimulated.

These and other embodiments which will be apparent to those of skill in the art upon reading the specification provide the art with methods for treatment and drug development for neural injuries caused by disease or trauma.

### BRIEF DESCRIPTION OF THE DRAWINGS

FIG. 1A-1B. Activated T cells mediate neurotoxicity via GrB. Peripheral blood mononuclear cells (PBMC) or sorted CD4⁺ or CD8⁺ cells were cultured for three days in Iscoves's modified Dulbecco's Medium +5% human serum. In parallel, these cells were incubated with anti-CD3/CD28 (cells:beads, 10:1) mAb-conjugated magnetic beads to induce polyclonal activation (Ac). Supernatants (sups) were then collected for GrB detection. Both activated CD4⁺ and CD8⁺ T cells released significant high amounts of GrB into the sups as determined by GrB immunoprecipitation (FIG. 1A). Cultured human fetal neurons were treated with culture supernatants (1:10 in Locke's buffer) from unsorted human T cells. Control cultures were treated with supernatants from unactivated T cells. To determine the role of GrB, activated T cell supernatant (AcT) was firstly immunodepleted with antibody against GrB (GrBAb) or control mouse IgG bound to protein A beads before treatment. Neurotoxicity was determined by calculating the percentage of cells positive with trypan blue 48 h later. Data represent mean±SEM of three replicates from three independent experiments. Image representative for three independent experiments is shown.

FIG. 2A-2F. GrB causes caspase-3 activation in neurons: (FIG. 2A) Untreated neurons show immunostaining for βIII tubulin (red) demonstrating normal morphology but are negative for caspase-3. (FIG. 2B-FIG. 2F) GrB treatment induced caspase-3 expression and apoptosis in some neurons. (FIG. 2B), a βIII tubulin positive neuron shows a fragment nucleus, indicating apoptosis. (FIG. 2C-FIG. 2F), a 13111 tubulin positive (FIG. 2C) neuron shows increased expression of caspase-3 (FIG. 2D) and a fragmented nucleus (FIG. 2E)

FIG. 3A-3C. GrB induced neurotoxicity is perforin and M6P receptor independent but mediated by Giα receptors and caspase dependent pathways. (FIG. 3A) Human neuronal cultures were treated with GrB (1 nM), human perforin (50 ng/ml) or with the combination of the two. (FIG. 3B), Human fetal neurons were treated with GrB in Locke's buffer for 48 h. Mannose-6-phosphate (M6P, 1 mM), pertussis toxin (PTX, 100 ng/ml) or caspase inhibitor Z-VAD-FMK (Z-VAD, 10 uM) were added 1 h prior to GrB treatment. Neurotoxicity was determined with trypan blue uptake assay. (FIG. 3C), cAMP levels were measured in neuronal cultures following GrB treatment. GrB treatment results in a significant decreases (P<0.01) in cAMP level at 5 min. Data represent mean±SEM of three replicates from three experiments.

FIG. 4-4B. disrupts calcium homeostasis in neurons. Primary neurons were exposed to GrB for 30 min before imaging of intracellular calcium using Fura-2AM. (FIG. 4A). Resting levels of cytosolic calcium were increased by treatment with 1.0-100 nM GrB. (FIG. 4B). SDF-1α (10 μM) evoked increases of cytosolic calcium were increased by pre-treatment of neurons with 10 nM GrB. * P<0.001.

FIG. 5 Antioxidants and GPI compounds protect neurons from GrB induced toxicity. Neuronal cultures were treated with GrB in Locke's buffer in the presence or absence of SOD/Catalase mimetic MnTMPyP (MnT, 10 μM, FIG. 5A), Trolox (Vitamin E, 10 μM), GPI-1046 (10 μM) which were added 1 h prior to GrB treatment. Neurotoxicity was then determined 48 hrs later with trypan blue uptake assay. Data are shown as mean±SEM of three replicates from one experiment, representing three independent experiments with similar results. *P<0.05, compared to control; #<0.05, compared to GrB alone treated groups.

FIG. 6 Schematic flow chart depicting the possible mechanism for GrB mediated activated T cells supernatant-induced neuronal damage. CD3/CD28 activated T cells release GrB into the supernatants. GrB induces neurotoxicity by both perforin dependent or independent mechanisms. As for the later, GrB activates PTX-sensitive Gi-coupled receptors by either direct binding, or by cleaved fragments binding or by direct cleavage. The activation of Gi-coupled receptor then results in decreased cAMP levels and elevated intracellular calcium levels, which may induce caspase-3 activation, leading to neuronal apoptosis. It is likely that oxidative stress also participates in the neurotoxicity and it is upstream of caspase activation.

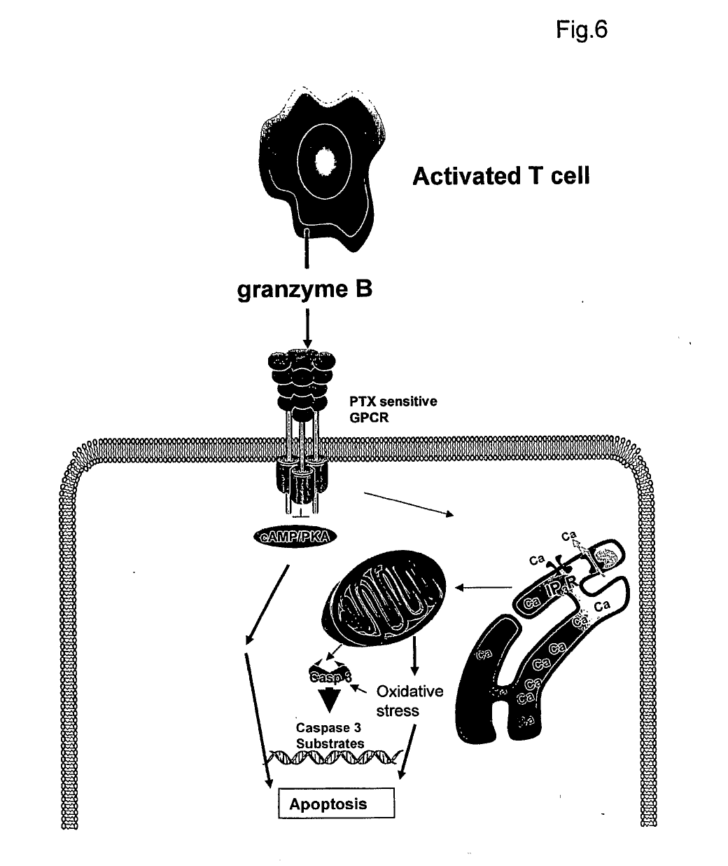

FIG. 7A-7H. Expression of Kv1.3 on neurons and toxicity by Granzyme B. Human fetal neuronal cultures were treated with GB (4 nM; FIGS. 7E-7H) or control with no GB (FIGS. 7A-7D) and immunostained with antisera to beta-tubulin (red; FIGS. 7A and 7E) Kv1.3 (green; FIGS. 7C and 7G) and Hoechst (blue; FIGS. 7B and 7F), 16 hours following treatment. Only minimal expression of Kv1.3 was noted in the untreated cultures, but a subpopulation of neurons in the GB treated cultures show marked expression of Kv1.3. These neurons also show evidence of DNA fragmentation of nuclear condensation suggestive of apoptotic features (inset). FIGS. 7D and 7H show merged images with immunostaining with both antisera and Hoechst.

FIG. 8A-8B Protection against Granzyme B neurotoxicity by Kv1.3 inhibitors. Human fetal neuronal cultures were treated with GB (4 nM) in the presence or absence of various K channel blockers. (FIG. 8A) Cell viability was determined by staining with CYTOQUANT™ blue and the staining was quantitated by a plate reader. A drop in optical density suggests neurotoxicity. rLq2 (Inward rectifier Kiv 1 blocker), rBeKm-1 (ERGI K channel blocker), dendrotoxin (voltage-gated Kv1.1 blocker) failed to protect against GB neurotoxicity while rTrtyustoxin (non-specific voltage gated K channel blocker) was protective. (FIG. 8B) Cell viability was determined by staining with trypan blue and the % of cells staining were counted which represent dead cells. A minimum of 500 cells was counted in each well and each treatment was done in triplicate. Data shows mean±SEM from three independent experiments. Margotoxin, a Kv1.3 channel blocker, protected against GB toxicity in a dose responsive manner, with significant protection at 1-10 nM.

FIG. 9A-9H. GrB treatment increased Kv1.3 expression in apoptotic Neuronal precursor cells (NPC). NPC cultures on cover slips were treated with GrB (4 nM; FIGS. 9B, 9D, 9F, and 9H) for 24 hours or controls without GrB (FIGS. 9A, 9C, 9E, and 9G). The cells were then fixed in 4% paraformaldehyde and immunostained with mAb against nestin (FIGS. 9A-9B) and polyclonal Ab against Kv1.3, followed by fluorescence-conjugated secondary Abs (FIGS. 9C-9D). Nuclei were stained with Hoechst 33342 (Calbiochem, 10 μM; FIGS. 9E-9F). The cells were imaged by confocal microscopy. As shown, more than 98% of the cells were positive for nestin and also moderately stained for Kv1.3 in the control cultures. In GrB treated cells, there was significant increase in Kv1.3 expression, especially in a subset of cells which showed bright and dense fluorescent stained nuclei with decreased nestin signal, indicating these cells may be undergoing apoptosis.

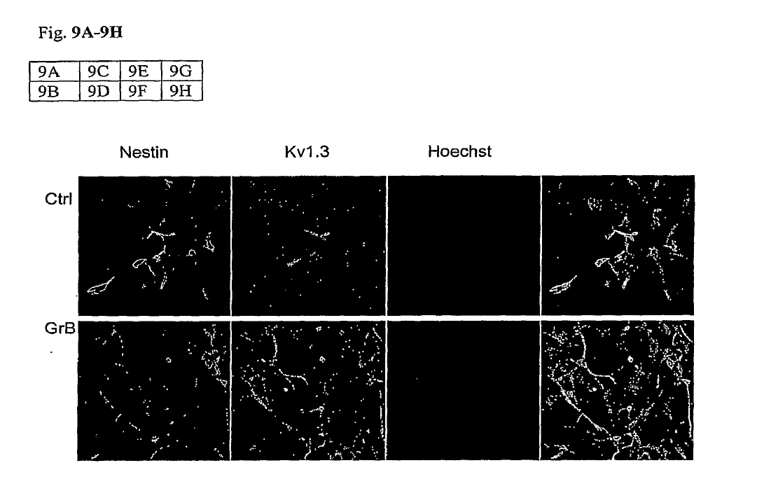

FIG. 10A-10B. Kv1.3 specific blocker attenuated GB-induced caspase-3 activation in NPC. NPC cultures were treated with GrB (4 nM) for 24 hours with/without pretreatment with pertussis toxin (PTX, 100 ng/ml; lane 3), Rock inhibitor (ROCK-I, 10 nM; lane 4) or Kv1.3 specific blocker margatoxin (MgTX, 10 nM; lane 5). Cell lysates were then collected and Western-blot was performed to determine the expression of Kv1.3. As seen in FIG. 10A, GB treatment induced caspase-3 activation, while pretreatment with PTX, ROCK-I and MgTX attenuated GB-induced caspase-3 activation, indicating Rho A, Kv1.3, as well as G-protein related pathways may mediate GB-induced apoptosis in NPC. Results from FIG. 10A are indicated quantitatively in FIG. 1013.

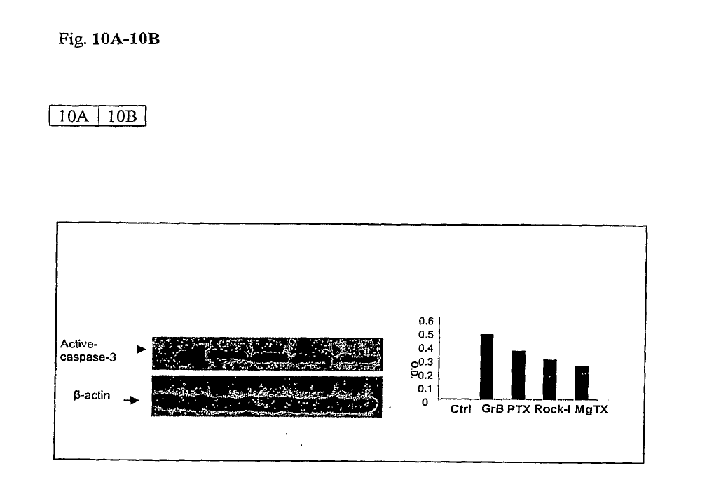

FIG. 11. Kv1.3 specific blocker enhanced neuronal differentiation in NPC. NPC cultures were treated with (FIGS. 11D-11F) or without (FIGS. 11A-11C) Kv1.3 specific blocker MgTX (10 nM) for 7 days in differentiating media. The cells were then fixed in 4% paraformaldehyde and immunostained with mAb against neuron specific βIII-tubulin (FIGS. 11A, 11D) and polyclonal Ab against astrocyte specific GFAP (FIGS. 11B, 11E), followed by fluorescence-conjugated second Abs. The cells were observed and images were taken under confocal microscopy. As shown here, Kv1.3 specific blocker MgTX treatment increased the number of βIII-tubulin positive cells and processes per cell, indicating Kv1.3 blocker may enhance neuronal differentiation. FIGS. 11C and 11F show the merged images.

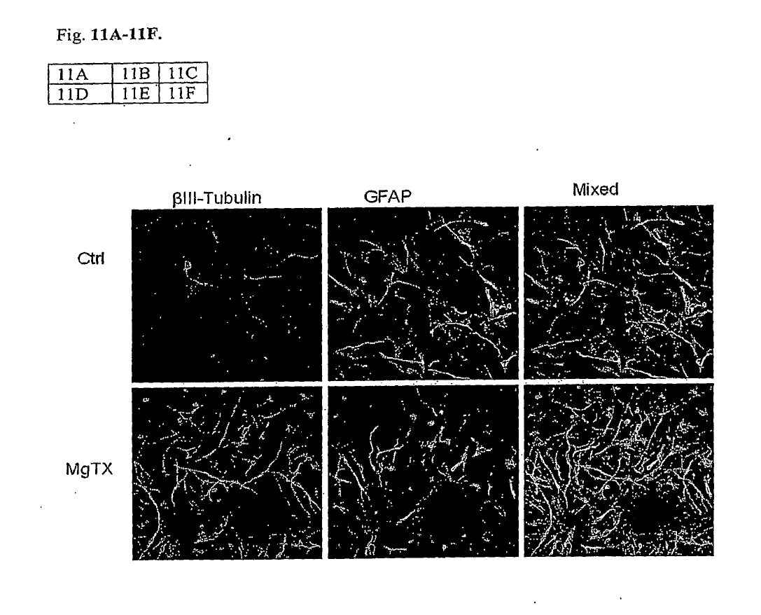

FIG. 12. Kv1.3 siRNA attenuated GrB-reduced neurite lengths. Cultured human fetal neurons were transfected with siRNA against Kv1.3 (GrB/KvSi) or a non-specific siRNA control (GB/NSI) prior to GrB treatment.

### DETAILED DESCRIPTION OF THE INVENTION

The inventors have discovered that expression of Kv1.3 channels is induced in neurons when they are damaged. Moreover, blockers of these channels or blockers of their expression protects neurons from damage and promotes neurite outgrowth. Thus treatment with blockers of function and/or expression of Kv1.3 is useful for neurological injuries, whether due to disease or traumatic injury.

Mammals which can be treated according to the present invention include without limitation humans, mice, rats, pigs, cows, dogs, cats, etc. Typically the animal will be one that comprises a suitable model for a human neurological disease or condition. Such diseases include both inflammatory (such as multiple sclerosis) and non-inflammatory neurological diseases, including, Alzheimer's Disease and other dementias, Parkinsonism such as Parkinson's Disease, Huntington's disease, amyotrophic lateral sclerosis, traumatic nerve injury such as from automobile accidents, falls, and sports-related activities, stroke. Other inflammatory neurological disorders which may be treated according to the present invention include Behcet's disease, Rasmussen's encephalitis, immune reconstitution syndrome in patients with HIV infection, and HTLV-1 associated myelopathy. Additional neuropathies caused by inflammation resulting from immune system activities rather than from direct damage by infectious organisms include acute inflammatory demyelinating neuropathy, better known as Guillain-Barré syndrome, chronic inflammatory demyelinating polyneuropathy (CIDP), multifocal motor neuropathy, whether chronic or acute.

Direct administration of an inhibitor or expression blocker to the CNS can be accomplished by any means known in the art, including but not limited to the cerebrospinal fluid via spinal catheter, to the striatum, intraventricular, intrathecal, intracerebral, and intraparenchymal injections. General administrations means of such agents include systemic administrations, including parenteral or oral administrations, such as intramuscular, intravenous, intraperitoneal, intranasal, intrabronchial, subcutaneous, and intradermal.

Suitable Kv1.3 channel blockers are those which are specific for Kv1.3 channels and do not affect other channels appreciably. Non-specific blockers can cause toxic side effects which are undesirable. Among the blockers that are known in the art are SHK22DAP, SL5, PAP1, khellinone, 8-methoxypsoralen, and 5-methoxypsoralen. See, e.g., U.S. application publication no. 2005026130. In addition to small molecule blockers, such as the naturally occurring toxins and derivatives described above, antibodies can also be used to block the activity of Kv1.3 channels. Antibodies against particular epitopes or a mixture of epitopes as found, inter glia in NP_{—}002223 (SEQ ID NO: 1) can be used. Any molecule comprising an antibody binding region can be used, including full antibodies, single chain variable regions, antibody fragments, antibody conjugates, etc. The antibodies may be monoclonal or polyclonal.

Inhibitors of expression of Kv1.3 channels may be any nucleic acid which functions via complementarity to the mRNA for Kv1.3. An exemplary human sequence for Kv1.3 cDNA is found in NM_{—}002232 (SEQ ID NO: 2). Suitable forms of nucleic acids including antisense molecules, antisense constructs, siRNA, etc., can be used. Such molecules are thought to function by degradation of nucleic acids so that transcription and/or translation of the specific mRNA is reduced. See Milhavet et al., “RNA intereference in biology and medicine,” Pharmacological Reviews, 55: 629-648, 2003.

Antisense constructs, antisense oligonucleotides, RNA interference constructs or siRNA duplex RNA molecules can be used to interfere with expression of Kv1.3. Typically at least 15, 17, 19, or 21 nucleotides of the complement of Kv1.3 mRNA sequence are sufficient for an antisense molecule. Typically at least 19, 21, 22, or 23 nucleotides of Kv1.3 are sufficient for an RNA interference molecule. Preferably an RNA interference molecule will have a 2 nucleotide 3′ overhang. If the RNA interference molecule is expressed in a cell from a construct, for example from a hairpin molecule or from an inverted repeat of the desired Kv1.3 sequence, then the endogenous cellular machinery will create the overhangs.

Antisense oligonucleotides can be administered. The antisense oligonucleotides typically will have modified chemical structures to enhance stability in the body. One such modified structure contains phosphorothioates in the phosphate backbone. Other modifications which reduce nuclease degradation and retain susceptibility to RNase H can also be used. For example, 2′-O-methyl nucleosides can be used, particularly on the 5′ and 3′ ends. The oligonucleotides can be complementary to Kv1.3 mRNA. Oligonucleotides can be complementary to various portions of the mRNA. The region surrounding the start codon may be targeted, as can splice sites, if present. Double stranded inhibitory RNA molecules can also be used. These are typically about 20-26 bases in length or preferably 20-23 bases in length. The molecules preferably contain 2-nucleotide 3′ overhangs.

siRNA molecules can be prepared by chemical synthesis, in vitro transcription, or digestion of long dsRNA by RNase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvatérra P, and Rossi J. (2002). Expression of small interfering RNAs targeted against HIV-1 rev transcripts in human cells. Nature Biotechnol. 20:500-505; Miyagishi M, and Taira K., (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

Antisense or RNA interference molecules can be delivered in vitro to cells or in vivo, e.g., to the CNS of a mammal. Typical delivery means known in the art can be used. For example, delivery to the CNS can be accomplished by intracerebral injections. Other modes of delivery can be used without limitation, including: intravenous, intramuscular, intraperitoneal, intraarterial, local delivery during surgery, endoscopic, subcutaneous, and per os. Vectors can be selected for desirable properties for any particular application. Vectors can be viral or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

Drug screening or testing can be performed with cells which are genetically modified to express Kv1.3 channels or with neurons or T cells which naturally express Kv1.3 channels. Natural Kv1.3-expressing cells can also be genetically modified to overexpress Kv1.3 channels. Cells do not ordinarily express these channels can be transfected with an expression construct which encodes Kv1.3 channels, permitting their expression in the cells which do not ordinarily do so. The expression construct can be any plasmid or viral vector, for example. The expression construct can include a promoter and optionally other transcriptional regulatory elements which are operably linked to permit expression under desired conditions or constitutively.

When screening test agents for potential as neuroprotective agents, one can assess ability of a test agent to reverse the cytological damage caused by Granzyme B on cells which express Kv1.3 channels. While T cells and neuronal cells naturally express such channels, other cells can be used if they are genetically manipulated to express such channels. Such cells may be more convenient to culture, more uniform, and present a simpler system than T cells or neuronal cells. The Granzyme B used in such assays can be a natural product secreted by activated T lymphocytes or can be a recombinant product. If a test agent reduces the damage or increases recovery of cell growth and/or development, one can identify it as a candidate neuroprotective agent. Reduction by a test agent is assessed using standard statistical measures of significance. Any standard statistical test can be applied to determine if the effect observed is significant. Identification involves articulating such potential, for example by recording the conclusion in a lab notebook, manuscript, computer, or other means of communication and information storage. Typically, further tests will be done to confirm the potential. Such tests might involve testing on T cells or neuronal cells which naturally express Kv1.3 channels. Other confirmatory tests might involve experimental animals in which a neurological disease or damage is induced. Finally, if tests in cultured cells and experimental animal models are encouraging, then the candidate neuroprotective agent can be tested in clinical settings on patients. An alternative type of confirmatory test would be to determine that the test agent actually works by inhibition of the Kv1.3 channel. Thus competition with other known inhibitors, such as Margotoxin, can be used to determine such a mode of action. Binding of the test agent directly to Kv1.3 channels can also be tested and determined.

Cell viability assessments can be performed by determining cells which are live or cells which are undergoing apoptosis. Hallmarks of apoptosis which can be readily determined include chromatin condensation, caspase activation, TUNEL assay, laddering of DNA, etc.

It is well known in the art that viability of a cell can be determined by contacting the cell with a dye and viewing it under a microscope. Viable cells can be observed to have an intact membrane and do not stain, whereas dying or dead cells having “leaky” membranes do stain. Incorporation of the dye by the cell indicates the death of the cell. The most common dye used in the art for this purpose is trypan blue. Viability of cells can also be determined by detecting DNA synthesis. Cells can be cultured in cell medium with labeled nucleotides, e.g., ³H thymidine. The uptake or incorporation of the labeled nucleotides indicates DNA synthesis. In addition, colonies formed by cells cultured in medium indicate cell growth and is another way to test viability of the cells.

Apoptosis is a specific mode of cell death recognized by a characteristic pattern of morphological, biochemical, and molecular changes. Cells going through apoptosis appear shrunken, and rounded; they also can be observed to become detached from culture dish. The morphological changes involve a characteristic pattern of condensation of chromatin and cytoplasm which can be readily identified by microscopy. When stained with a DNA-binding dye, e.g., H33258, apoptotic cells display classic condensed and punctate nuclei instead of homogeneous and round nuclei.

A hallmark of apoptosis is endonucleolysis, a molecular change in which nuclear DNA is initially degraded at the linker sections of nucleosomes to give rise to fragments equivalent to single and multiple nucleosomes. When these DNA fragments are subjected to gel electrophoresis, they reveal a series of DNA bands which are positioned approximately equally distant from each other on the gel. The size difference between the two bands next to each other is about the length of one nucleosome, i.e., 120 base pairs. This characteristic display of the DNA bands is called a DNA ladder and it indicates apoptosis of the cell. Apoptotic cells can be identified by flow cytometric methods based on measurement of cellular DNA content, increased sensitivity of DNA to denaturation, or altered light scattering properties. These methods are well known in the art and are within the contemplation of the invention.

Abnormal DNA breaks are also characteristic of apoptosis and can be detected by any means known in the art. In one embodiment, DNA breaks are labeled with biotinylated dUTP (dUTP). Cells are fixed and incubated in the presence of biotinylated dUTP with either exogenous terminal transferase (terminal DNA transferase assay; TdT assay) or DNA polymerase (nick translation assay; NT assay). The biotinylated dUTP is incorporated into the chromosome at the places where abnormal DNA breaks are repaired, and are detected with fluorescein conjugated to avidin under fluorescence microscopy.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Cells and Cell Cultures

Peripheral blood mononuclear cells were isolated from three different healthy human donors by standard ficoll separation from heparinized whole blood. CD4+ and CD8+ cells were isolated by negative selection using MACS beads (Miltenyi Biotec). T cell subsets were incubated at 37° C. in Iscove's modified Dulbecco's medium supplemented with 5% human serum and activated (Ac) by placing on plates coated with 1 ug/mL of anti-CD3 and 1 ug/mL of soluble anti-CD28 for 72 hours in culture. Culture supernatants were then collected and incubated (1:10 dilution) with human fetal neurons.

Human fetal neurons were cultured as previously described (35). Briefly, human fetal brain specimens of 12-17 weeks gestation were obtained in accordance with NIH guidelines. The tissues were then triturated after removing the meninges. Cells were then cultured in T75 flasks in opti-MEM with 5% FBS, 0.5% N2 supplement and 1% antibiotics. Neurons were collected by carefully shaking the flask at least 1 month later. Cells were then seeded at 1×10⁵/ml in 96 well plates for 1 week before treatment. These cultures contain 70-80% neurons, <5% microglia and the remaining cells are astrocytes as determined by immunostaining for microtubule associated antigen (MAP-2), CD68 and glial fibrillary acidic protein (GFAP) respectively.

Enriched human fetal astroglia were cultured as previously described. Briefly, human fetal brain specimens of 12-17 weeks gestation were triturated after removal of the meninges. Cells were then cultured in T75 flasks in DMEM with 10% FBS and 1% antibiotics for at least 1 month. After shaking at 180 rpm for 1 h, cells were separated with trypsin/EDTA. Cells were then seeded at 1×10⁵/ml in 96 well plates for 1 week before treatment. >95% of these cells were immunostained for GFAP.

### Detection of GrB

To determine if activated T cells release GrB extracellularly, T cell culture supernatants were collected and clarified by centrifuging at 9000 rpm for 10 min. The pellet was discarded. GrB in the supernatants were then determined by Western-blot. For Western-blot, 50 μL of the supernatant was concentrated by precipitation with tricholoroacetic acid. The pellet was then mixed with SDS sample buffer and boiled for 5 min. Samples were resolved on a 15% Tris-glycine polyacrylamide gel. Following transfer of proteins to a polyvinylidene difluoride (PVDF) membrane, the blot was probed with a monoclonal antibody to GrB. Immunoreactive bands were visualized by electrochemiluminescence (Amersham). The intensity of the signal was quantified using a densitometer.

### Immunodepletion of GrB

To immunodeplete GrB from T cell supernatants, the supernatants were incubated 1:1 with pre-swollen protein G sepharose (Pharmacia) for 2 h at 4° C., a step taken to eliminate proteins in the lysate which may bind non-specifically to the protein G. The mix was subsequently spun and the supernatant was incubated at 4° C. overnight with anti-GrB or an isotype-matched control antibody. This mix was incubated for 2 h with protein G sepharose and filtered through a column. The supernatant was then used to treat neuronal cultures. All incubations (antibody and protein G) were performed on a rotary table at 4° C., and all centrifugations were performed using a desktop Eppendorf centrifuge at 4° C. for 5 min at maximum speed (9000 g).

### Neurotoxicity Assays

Neurotoxicity was evaluated by using MTT and trypan blue uptake assays. For MTT assay, collected neurons were cultured at 1×10⁵/ml in Locke's buffer in 96-well plates. T cells culture supernatants (1:10 to 1:100 dilution) were then added and cultured for 44 hours. MTT (5 mg/ml) was added to the cultures and cells were incubated for another 4 hours. Dimethyl sulfoxide (DMSO; 50%) was added to dissolve the formazan and the optical density (OD) value was detected at 590 nm.

For trypan blue uptake, neuronal cells were seeded at 1×10⁵/ml and incubated for 1 week in 96-well plates before treatment. After adding the reagents (GrB (0.5-4 nM; Calbiochem), perform (50 ng/ml), MnTMPyP (SOD/Catalase mimetic; 10 uM; Calbiochem), pertussis toxin (PTX)(100 ng/ml; Calbiochem), Z-VAD-fmk (10 uM; BIOMOL), D-mannose 6-phosphate (1 mM; Calbiochem), stromal derived factor 1-α (SDF-1α;), trolox (analogue of vitamin E; 10 μM; Sigma), immunophilin 3-(3-pyridyl)-1-propyl (2S)-1-(3,3-dimethyl-1,2-dioxopentyl)-2-pyrrollidinecarboxylate (10 μM; GPI-1046; gifted by Guilford Pharmaceuticals Inc.) the cells were incubated for another 48 hours. The cells were then stained with trypan blue for 5 min, washed with phosphate buffered saline, pH7.4 (PBS) and fixed with 4% paraformaldehyde. Trypan blue positive and negative cells were counted in three pre-determined fields. Approximately 200 cells were counted in each well. Each experiment was done in triplicate wells and mean and SEM were calculated from at least three independent experiments.

### Caspase-3 Activation

Human fetal neuron cultures on cover slips were treated with GrB (1 nM) for 24 h. Caspase-3 and βIII tubulin expression in neurons was determined by immunocytochemistry. Briefly, cells were fixed with 4% paraformaldehyde for 5 min, and then blocked with 3% FBS in PBS for 20 min. Polyclonal caspase-3 antiserum (1:500) and monoclonal anti-BIII tubulin (1 μg/ml; Promega, Madison. WI) antibody were then applied and cover slips incubated overnight at 4° C. After washing with PBS, the cover slips were incubated with secondary antibodies (1:200 Donkey anti-rabbit IgG Alexa Fluor 594 and anti-mouse IgG Alexa Fluor 488, Molecular Probes, Eugene, Oreg.) for 2 h. Hochest 33258 (10 μM) was added at the last half an hour to stain the nucleus. The cells were imaged by confocal microscopy.

### Cyclic AMP Assay

Human fetal neuronal cultures were seeded at 5×10⁵/ml in 12 well plates for 1 week before treatment. Cultures were then treated with GrB (1 nM) in Locke's buffer for 0-30 min. After removing the media the cells were treated with 100 μl of 0.1 M HCl for 10 min to achieve cell lysis. The lysate was centrifuged at 600 g for 10 min and the supernatant was used directly for the cyclic AMP assay. Cyclic AMP competitive ELISA kit (ENDOGEN, Rockford, Ill.) was used according to manufacturer's directions.

### Calcium Imaging

Cytosolic calcium ([Ca²⁺]_{c}) was determined using the ratiometric calcium probe Fura-2/AM using methods similar to those previously described (36). Cells were incubated in 2 μM Fura-2/AM for 20 min at 37° C. in media and washed with Locke's Buffer (154 mM NaCl, 3.6 mM NaHCO₃, 5.6 mM KCl, 1 mM MgCl₂, 5 mM HEPES, 2.3 mM CaCl₂, 10 mM glucose; pH 7.4) to remove extracellular Fura-2/AM. Fura-2 loaded cells were placed into a open bath chamber and maintained at 37° C. (Series 20 open perfusion chamber and TC-344B temperature controller; Warner Instruments. Hamden, Conn.). Buffer flowed over the cells at the rate of ˜2 ml/min using a VC-6 perfusion control system with a multi-input manifold that minimized dead space, allowing for a rapid change between buffer and buffer containing drug. Cells were alternatively excited at 340 and 380 nm by a monochrometer and emission was recorded at 510 with software from Intracellular Imaging (Intracellular Imaging Inc., Cincinnati, Ohio.) 340/380 nM ratios were converted to nM [Ca²⁺]_{c} using curve fitting software and calcium reference standards (Molecular Probes).

### EXAMPLE 2

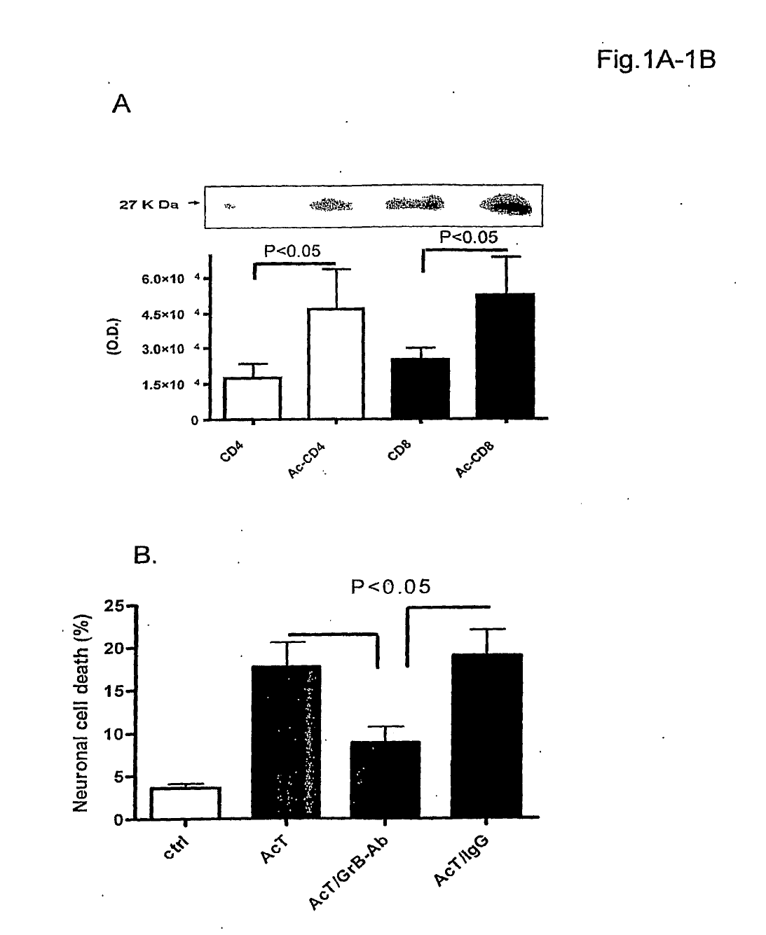

Activated T cells release GrB Which is neurotoxic. To determine if activated T cells release neurotoxic soluble factors, we exposed cultured human fetal neurons to supernatants from purified T cells that had been activated with anti-CD3 and anti-CD28 antibodies and assessed neuronal viability by either a MTT assay or by trypan blue exclusion. We found that the culture supernatants from activated T cells induced significant toxicity to neurons compared to unstimulated T cells and the toxicity was more prominent with supernatants from activated CD8+ cells (Data not shown). These observations indicate that activated T-cells may release soluble factors to induce neuronal toxicity. Because GrB is an important factor in mediating T cell-induced cytotoxicity, we examined the production of GrB in the cultured T cell supernatants by semiquantitative western-blot analysis and ELISA. As shown in FIG. 1A, all the supernatants contained GrB. However, activation of both CD4+ and CD8+ T cells increased release of GrB significantly compared to the corresponding controls (P<0.05). To further determine if the released GrB was responsible for the neurotoxicity, we immunodepleted the GrB from the supernatants and found that neurotoxicity of the supernatants was significantly attenuated, however when the supernatants were similarly treated with an isotype control antibody, no loss of neurotoxicity was noted, clearly demonstrating that the neurotoxicity of the T cell supernatants was at least in part due to GrB. (FIG. 1B).

### EXAMPLE 3

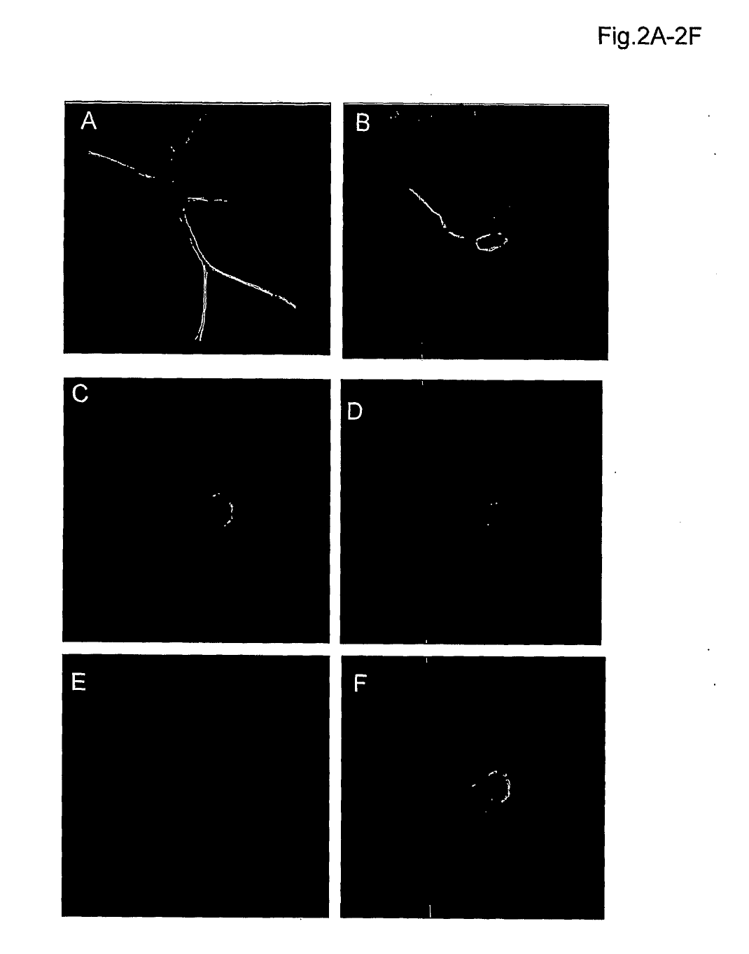

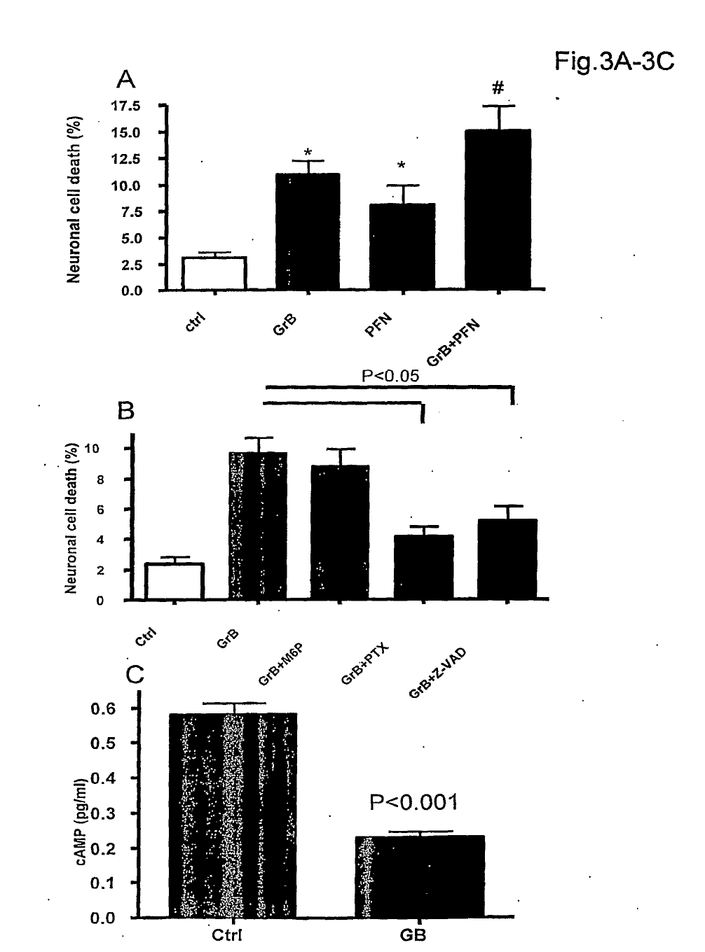

Recombinant GrB induces toxicity in neurons but not in astroglia. To further confirm that GrB could induce neurotoxicity we used recombinant GrB (0.5 to 4 nM) and found that 1 nM was the minimum concentration needed to cause significant neurotoxicity as demonstrated by caspase-3 activation and nuclear fragmentation suggestive of apoptosis (FIG. 2). We next determined the effect of perform on GrB induced neurotoxicity. We used threshold toxic concentrations of perform (50 ng/ml) for these experiments. Some enhancement of GrB neurotoxicity may be present but the enhancement was not statistically significant (P>0.05). Clearly no synergistic effects were seen with GrB and perforin (FIG. 3A). These observations suggest that GrB alone may be sufficient to cause neurotoxicity. We next determined if the GrB mediated toxicity could also occur in astrocytes. Human astrocyte cultures were similarly treated with recombinant GrB (dose-1-4 nM) and monitored for neurotoxicity. No evidence of cell death was noted in these cultures (data not shown). This suggests that GrB toxicity is specific for a subpopulation of neurons.

### EXAMPLE 4

GrB-induced neurotoxicity is independent of mannose-6 phosphate receptor but is mediated by Giα/Go coupled receptors and caspase dependent pathways. Since GrB may enter cells in a perforin independent manner via interactions with the mannose-6-phosphate receptor (37), we pretreated the cells with 10 mM mannose-6-phosphate followed by GrB (1 nM). Mannose-6-phosphate was unable to inhibit GrB-induced neurotoxicity suggesting that GrB-mediated neurotoxicity is independent of both perforin and mannose-6-phosphate receptor (FIG. 3B). However, GrB-mediated neurotoxicity could be significantly (P<0.05) blocked by Z-VAD-fmk (FIG. 3B), a broad spectrum caspase inhibitor suggesting that GrB-induces neuronal toxicity via activation of caspase dependent apoptotic pathways. Interestingly, GrB induced neurotoxicity could also be blocked by 10 μM pertussis toxin (PTX) (FIG. 3B) suggesting a role for act/Go coupled receptors in apoptotic pathway mediated neuronal cell death. Consistent with its ability to act on PTX sensitive receptors, GrB-stimulates a decrease in cAMP. cAMP levels were measured in neuronal cultures following GrB treatment. Decreases in cAMP levels occurred in a time dependent manner with significant decreases at 5 min (FIG. 3C).

### EXAMPLE 5

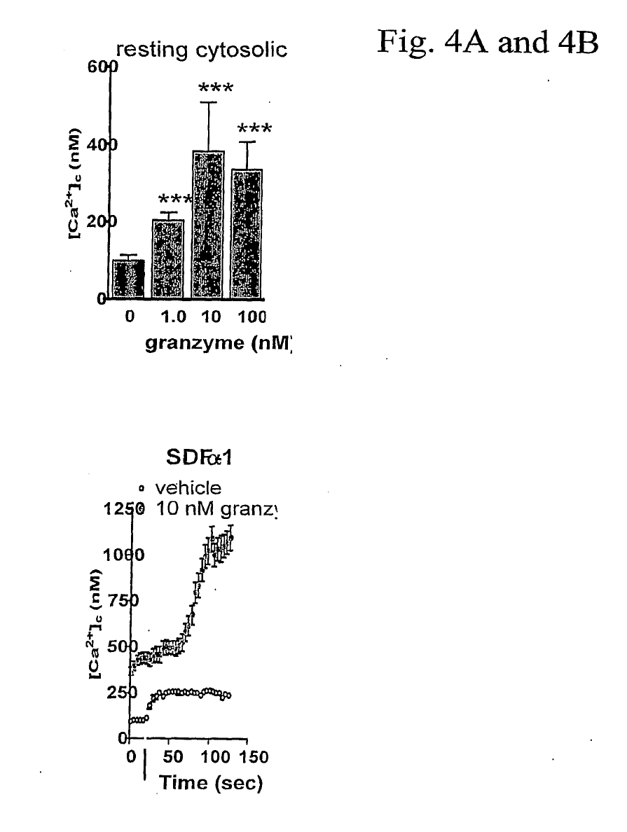

GrB disrupts calcium homeostasis in neurons. To determine if GrB could disrupt calcium homeostasis, purified GrB was applied onto neurons and [Ca²⁺]_{c} was measured in real time. A dose-dependent increase in the basal level of [Ca²⁺]_{c} was noted within 30 min (FIG. 4A). At the lowest concentration tested, −1 nM of GrB doubled the resting concentration of [Ca²⁺]_{c}. Higher dose of GrB increased resting [Ca²⁺]_{c} four fold (FIG. 4A). Because elevated [Ca²⁺]_{c} levels can result in endoplasmic reticulum (ER) calcium overload and neuronal death, we determined if GrB enhanced IP3-mediated ER calcium release using, SDF-1α, a G-protein coupled receptor that stimulates ER calcium release via the Gi α subunits (38). We found that pretreatment with GrB resulted in a marked increase of SDF-1α-evoked ER calcium release from a peak increase of 250 nM in vehicle treated cultures to 1000 nM in cultures pre-treated with GrB (FIG. 4B). The ability of GrB to potentiate the response of SDF-1 is consistent with its ability to stimulate a Gi protein coupled receptor.

### EXAMPLE 6

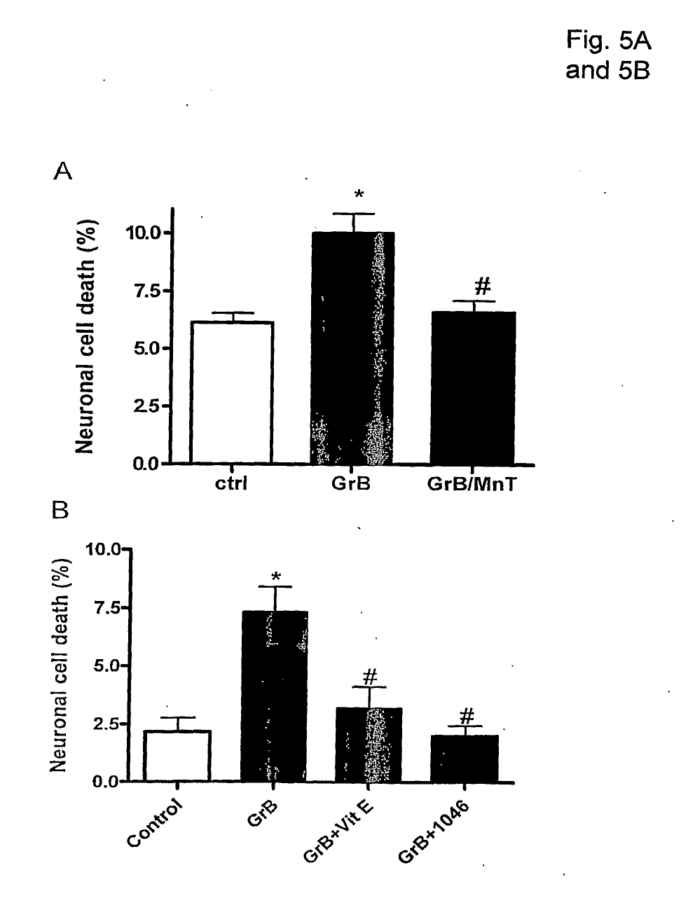

Attenuation of GrB-induced neurotoxicity with SOD/Catalase mimetic, vitamin E and neuroimmunophilin. To screen for possible agents that could protect against GrB-induced neurotoxicity, we pretreated the cultures with 10 μM MnTMPyP, a SOD/Catalase mimetic, and found that it significantly blocked the neurotoxicity (FIG. 5A, P<0.05). Similarly, we found that 10 μM trolox, analog of vitamin E, and neuroimmunophilin GPI-1046 also blocked GrB neurotoxicity (P<0.05) (FIG. 5B). GPI-1046 is an agent with both neuroprotective and neurotrophic effects but the exact mechanism of action remains unknown.

### EXAMPLE 7

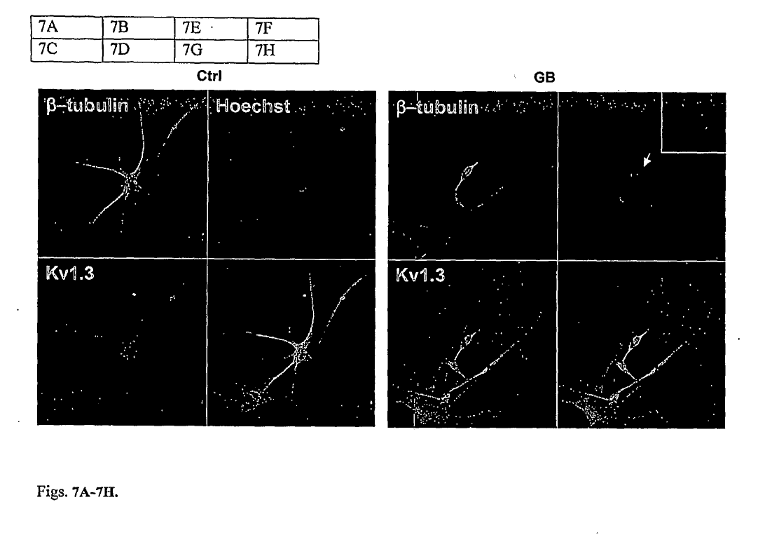

Expression of voltage gated channel, Kv1.3, in neurons. We discovered that that similar to T cells small basal levels of Kv1.3 channel were expressed on neurons. However, upon induction of neuronal injury by treatment with GB, there was increased expression of Kv1.3 channel in a subpopulation of cells. These cells showed retraction of neurites and nuclear fragmentation and condensation. Similar increases in Kv1.3 immunostaining in neurons were also noted upon treatment with supernatants from activated T cells (FIG. 7A-7H). The increased expression of Kv1.3 on injured neurons suggests that this might be a good target for neuroprotection without affecting the normal functions of neurons.

### EXAMPLE 8

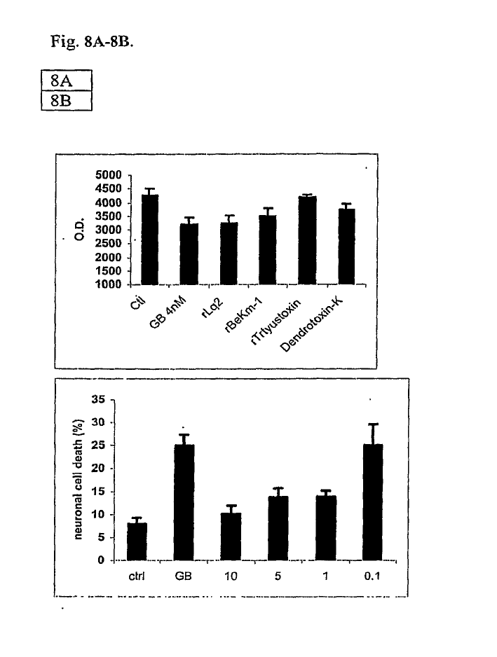

Pharmacological blockers of Potassium gated-channel Kv1.3 prevent Granzyme B (GB)-induced neurotoxicity. While the ability of Kv1.3 blockers to block T cell activation has been previously shown by several groups (Hanson et al, 1999. UK-78,282, a novel piperidine compound that potently blocks the Kv1.3 voltage-gated potassium channel and inhibits human T cell activation. Br J Pharmacol 126: 1707-16; Kalman et al, 1998. ShK-Dap22, a potent Kv1.3-specific immunosuppressive polypeptide. J Biol Chem 273: 32697-707; Nguyen et al, 1996. Novel nonpeptide agents potently block the C-type inactivated conformation of Kv1.3 and suppress T cell activation. Mol Pharmacol 50: 1672-9.), its potential role in preventing neurotoxicity has not been explored. We explored the potential role of several different K channel blockers in preventing GB-induced neurotoxicity and found that only compounds that blocked the Kv1.3 channel were effective in blocking GB-induced neurotoxicity (FIG. 8A-8B). This is a novel observation, because most compounds available to date that prevent T cell activation either have no effect on neurons or often cause neurotoxicity (Lischke et al, 2004. Cyclosporine-related neurotoxicity in a patient after bilateral lung transplantation for cystic fibrosis. Transplant Proc 36: 2837-9; Serkova et al, 2004. Biochemical mechanisms of cyclosporine neurotoxicity. Mol Interv 4: 97-107; Yamauchi et al, 2005. Cyclosporin A aggravates electroshock-induced convulsions in mice with a transient middle cerebral artery occlusion. Cell Mol Neurobiol 25: 923-8). Similarly, most neuroprotective compounds either have no effect on T cells or may enhance their activation due to their anti-apoptotic properties (Weinreb et al, 2005. Novel Neuroprotective Mechanism of Action of Rasagiline Is Associated with Its Propargyl Moiety: Interaction of Bcl-2 Family Members with PKC Pathway. Ann N Y Acad Sci 1053: 348-55). Alternatively, some neuroprotective compounds may inhibit T cell migration to the brain (Kao et al, 2005. Neuroprotection by tetramethylpyrazine against ischemic brain injury in rats. Neurochem Int). Kv1.3 thus represent a unique target where both neuroprotection and prevention of T cell activation can be accomplished. We now need to determine how 013 activates the Kv1.3 channel, what the relationship of this channel is with the GRCP and how activation of this channel triggers neuronal cell death.

### EXAMPLE 9

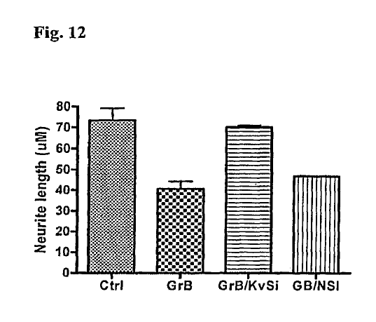

Inhibition of expression of Kv1.3 attenuates GrS-reduced neurite length. 60% confluent human fetal neurons cultured on poly-D-lysine coated cover slips were transfected with siRNA against Kv1.3 (25 nM final concentration) using lipofectamine (Invitrogen, USA) 24 hr prior to GrB treatment (4 nM). Another 24 hr later, cover slips were collected and fixed for γ-III-tubulin immunostaining. The neurite lengths in at least 9 pre-selected fields in each group were measured using Open-lab software. The average of neurite lengths from three experiments was presented (except GB/NSI, which is from a single experiment). The result shows Kv1.3 siRNA (see siRNA #3 below) attenuated GrB-reduced neurite length while a non-specific control siRNA (NSI) did not. See FIG. 12.

v1.3 siRNA sequences tested include:

(SEQ ID NO: 3)

| | |
|---|---|
| 3: sense sequence: | GGAAAACCACUGUUUGAAUtt; |

(SEQ ID NO: 4)

| | |
|---|---|
| Antisense Sequence: | AUUCAAACAGUGGUUUUCCtt; |

(SEQ ID NO: 5)

| | |
|---|---|
| 4: Sense: | GCAAUCCCAGUACAUGCACtt; |

(SEQ ID NO: 6)

| | |
|---|---|
| Antisense: | GUGCAUGUACUGGGAUUGCtc; |

(SEQ ID NO: 7)

| | |
|---|---|
| 5: Sense: | GCAUUAGACUAACAGAUUCtt; |

(SEQ ID NO: 8)

| | |
|---|---|
| Antisense: | GAAUCUGUUAGUCUAAUGCtt; |

(SEQ ID NO: 9)

| | |
|---|---|
| 6: Sense: | CCGAUGUUUAAUAUGUGAUtt; |

(SEQ ID NO: 10)

| | |
|---|---|
| Antisense: | AUCACAUAUUAAACAUCGGtg. |

### REFERENCES

The disclosure of each reference cited is expressly incorporated herein.
1. Zivadinov, R., Bagnato, F., Nasuelli, D., Bastianello, S., Bratina, A., Locatelli, L., Watts, K., Finamore, L., Grop, A., Dwyer, M., Catalan, M., Clemenzi, A., Millefiorini, E., Bakshi, R., and Zorzon, M. (2004) Short-term brain atrophy changes in relapsing-remitting multiple sclerosis. J Neurol Sci 223, 185-193
2. Redmond, I. T., Barbosa, S., Blumhardt, L. D., and Roberts, N. (2000) Short-term ventricular volume changes on serial MRI in multiple sclerosis. Acta Neurol Scand 102, 99-105
3. Brex, P. A., Jenkins, R., Fox, N. C., Crum, W. R., O'Riordan, J. I., Plant, G. T., and Miller, D. H. (2000) Detection of ventricular enlargement in patients at the earliest clinical stage of MS. Neurology 54, 1689-1691
4. Leist, T. P., Gobbini, M. I., Frank, J. A., and McFarland, H. F. (2001) Enhancing magnetic resonance imaging lesions and cerebral atrophy in patients with relapsing multiple sclerosis. Arch Neurol 58, 57-60
5. Zivadinov, R., Sepcic, J., Nasuelli, D., De Masi, R., Bragadin, L. M., Tommasi, M. A., Zambito-Marsala, S., Moretti, R., Bratina, A., Ukmar, M., Pozzi-Mucelli, R. S., Grop, A., Cazzato, G., and Zorzon, M. (2001) A longitudinal study of brain atrophy and cognitive disturbances in the early phase of relapsing-remitting multiple sclerosis. J Neurol Neurosurg Psychiatry 70, 773-780
6. Kalkers, N. F., Ameziane, N., Bot, J. C., Minneboo, A., Polman, C. H., and Barkhof, F. (2002) Longitudinal brain volume measurement in multiple sclerosis: rate of brain atrophy is independent of the disease subtype. Arch Neurol 59, 1572-1576
7. Dalton, C. M., Brex, P. A., Jenkins, R., Fox, N. C., Miszkiel, K. A., Crum, W. R., O'Riordan, J. I., Plant, G. T., Thompson, A. J., and Miller, D. H. (2002) Progressive ventricular enlargement in patients with clinically isolated syndromes is associated with the early development of multiple sclerosis. J Neural Neurosurg Psychiatry 73, 141-147
8. Fox, N. C., Jenkins, R., Leary, S. M., Stevenson, V. L., Losseff, N. A., Crum, W. R., Harvey, R. J., Rossor, M. N., Miller, D. H., and Thompson, A. J. (2000) Progressive cerebral atrophy in MS: a serial study using registered, volumetric MRI. Neurology 54, 807-812
9. Zivadinov, R., Rudick, R. A., De Masi, R., Nasuelli, D., Ukmar, M., Pozzi-Mucelli, R. S., Grop, A., Cazzato, G., and Zorzon, M. (2001) Effects of IV methylprednisolone on brain atrophy in relapsing-remitting MS. Neurology 57, 1239-1247
10. Hardmeier, M., Wagenpfeil, S., Freitag, P., Fisher, E., Rudick, R. A., Kooijmans-Coutinho, M., Clanet, M., Radue, E. W., and Kappos, L. (2003) Atrophy is detectable within a 3-month period in untreated patients with active relapsing remitting multiple sclerosis. Arch Neurol 60, 1736-1739
11. Dalton, C. M., Miszkiel, K. A., Barker, G. J., MacManus; D. G., Pepple, T. I., Panzara, M., Yang, M., Hulme, A., O'Connor, P., and Miller, D. H. (2004) Effect of natalizumab on conversion of gadolinium enhancing lesions to T1 hypointense lesions in relapsing multiple sclerosis. J Neurol 251, 407-413
12. Comi, G., and Moiola, L. (2002) Glatiramer acetate. Neurologia 17, 244-258
13. Trapp, B. D., Peterson, J., Ransohoff, R. M., Rudick, R., Mork, S., and Bo, L. (1998) Axonal transection in the lesions of multiple sclerosis. N Engl J Med 338, 278-285
14. Bjartmar, C., Kinkel, R. P., Kidd, G., Rudick, R. A., and Trapp, B. D. (2001) Axonal loss in normal-appearing white matter in a patient with acute MS. Neurology 57, 1248-1252
15. Bitsch, A., Schuchardt, J., Bunkowski, S., Kuhlmann, T., and Bruck, W. (2000) Acute axonal injury in multiple sclerosis. Correlation with demyelination and inflammation. Brain 123 (Pt 6), 1174-1183
16. Peterson, J. W., Bo, L., Mork, S., Chang, A., and Trapp, B. D. (2001) Transected neurites, apoptotic neurons, and reduced inflammation in cortical multiple sclerosis lesions. Ann Neurol 50, 389-400
17. Bien, C. G., Bauer, J., Deckwerth, T. L., Wiendl, H., Deckert, M., Wiestler, O. D., Schramm, J., Elger, C. E., and Lassmann, H. (2002) Destruction of neurons by cytotoxic T cells: a new pathogenic mechanism in Rasmussen's encephalitis. Ann Neurol 51, 311-318
18. Miller, R. F., Isaacson, P. G., Hall-Craggs, M., Lucas, S., Gray, F., Scaravilli, F., and An, S. F. (2004) Cerebral CD8+ lymphocytosis in HIV-1 infected patients with immune restoration induced by HAART. Acta Neuropathol (Berl) 108, 17-23
19. Lovett-Racke, A. E., Martin, R., McFarland, H. F., Racke, M. K., and Utz, U. (1997) Longitudinal study of myelin basic protein-specific T-cell receptors during the course of multiple sclerosis. J Neuroimmunol 78, 162-171_{.}
20. Wulff, H., Calabresi, P. A., Allie, R., Yun, S., Pennington, M., Beeton, C., and Chandy, K. G. (2003) The voltage-gated Kv1.3 K(+) channel in effector memory T cells as new target for MS. J Clin Invest 111, 1703-1713
21. Scholz, C., Patton, K. T., Anderson, D. E., Freeman, G. J., and Hafler, D. A. (1998) Expansion of autoreactive T cells in multiple sclerosis is independent of exogenous B7 costimulation. J Immunol 160, 1532-1538
22. Medana, I., Martinic, M. A., Wekerle, H., and Neumann, H. (2001) Transection of major histocompatibility complex class I-induced neurites by cytotoxic T lymphocytes. Am J Pathol 159, 809-815
23. Neumann, H. (2003) Molecular mechanisms of axonal damage in inflammatory central nervous system diseases. Curr Opin Neural 16, 267-273
24. Giuliani, F., Goodyer, C. G., Antel, J. P., and Yong, V. W. (2003) Vulnerability of human neurons to T cell-mediated cytotoxicity. J Immunol 171, 368-379
25. Hoftberger, R., Aboul-Enein, F., Brueck, W., Lucchinetti, C., Rodríguez, M., Schmidbauer, M., Jellinger, K., and Lassmann, H. (2004) Expression of major histocompatibility complex class I molecules on the different cell types in multiple, sclerosis lesions. Brain Pathol 14, 43-50
26. Neumann, H., Medana, I. M., Bauer, J., and Lassmann, H. (2002) Cytotoxic T lymphocytes in autoimmune and degenerative CNS diseases. Trends Neurosci 25, 313-319
27. Bauer, J., Bien, C. G., and Lassmann, H. (2002) Rasmussen's encephalitis: a role for autoimmune cytotoxic T lymphocytes. Curr Opin Neural 15, 197-200
28. Raja, S. M., Metkar, S. S., and Froelich, C. J. (2003) Cytotoxic granule-mediated apoptosis: unraveling the complex mechanism. Curr Opin Immunol 15, 528-532
29. Froelich, C. J., Orth, K., Turbov, J., Seth, P., Gottlieb, R., Babior, B., Shah, G. M., Bleackley, R. C., Dixit, V. M., and Hanna, W. (1996) New paradigm for lymphocyte granule-mediated cytotoxicity. Target cells bind and internalize granzyme B, but an endosomolytic agent is necessary for cytosolic delivery and subsequent apoptosis. J Biol Chem 271, 29073-29079
30. Trapani, J. A., Sutton, V. R., Thia, K. Y., Li, Y. Q., Froelich, C. J., Jans, D. A., Sandrin, M. S., and Browne, K. A. (2003) A clathrin/dynamin- and mannose-6-phdsphate receptor-independent pathway for granzyme B-induced cell death. J Cell Biol 160, 223-233
31. Adrain, C., Murphy, B. M., and Martin, S. J. (2005) Molecular ordering of the caspase activation cascade initiated by the cytotoxic T lymphocyte/natural killer (CTL/NK) protease granzyme B. J Biol Chem 280, 4663-4673
32. Pinkoski, M. J., Waterhouse, N. J., Heibein, J. A., Wolf, B. B., Kuwana, T., Goldstein, J. C., Newmeyer, D. D., Bleackley, R. C., and Green, D. R. (2001) Granzyme B-mediated apoptosis proceeds predominantly through a Bcl-2-inhibitable mitochondrial pathway. J Biol Chem 276, 12060-12067
33. Cartron, P. F., Juin, P., Oliver, L., Martin, S., Meflah, K., and Vallette, F. M. (2003) Nonredundant role of Bax and Bak in Bid-mediated apoptosis. Mol Cell Biol 23, 4701-4712
34. Trapani, J. A., Jans, D. A., Jans, P. J., Smyth, M. J., Browne, K. A., and Sutton, V. R. (1998) Efficient nuclear targeting of granzyme B and the nuclear consequences of apoptosis induced by granzyme B and perforin are caspase-dependent, but cell death is caspase-independent. J Biol Chem 273, 27934-27938
35. Magnuson, D. S., Knudsen, B. E., Geiger, J. D., Brownstone, R. M., and Nath, A. (1995) Human immunodeficiency virus type 1 tat activates non-N-methyl-D-aspartate excitatory amino acid receptors and causes neurotoxicity. Ann Neural 37, 373-380
36. Haughey, N. J., Holden, C. P., Nath, A., and Geiger, J. D. (1999) Involvement of inositol 1,4,5-trisphosphate-regulated stores of intracellular calcium in calcium dysregulation and neuron cell death caused by HIV-1 protein tat. J Neurochem 73, 1363-1374
37. Motyka, B., Korbutt, G., Pinkoski, M. J., Heibein, J. A., Caputo, A., Hobman, M., Barry, M., Shostak, I., Sawchuk, T., Holmes, C. F., Gauldie, J., and Bleackley, R. C. (2000) Mannose 6-phosphate/insulin-like growth factor II receptor is a death receptor for granzyme B during cytotoxic T cell-induced apoptosis. Cell 103, 491-500
38. Lazarini, F., Casanova, P., Tham, T. N., De Clercq, E., Arenzana-Seisdedos, F., Baleux, F., and Dubois-Dalcq, M. (2000) Differential signalling of the chemokine receptor CXCR4 by stromal cell-derived factor 1 and the HIV glycoprotein in rat neurons and astrocytes. Eur J Neurosei 12, 117-125
39. Kuhlmann, T., Lingfeld, G., Bitsch, A., Schuchardt, J., and Bruck, W. (2002) Acute axonal damage in multiple sclerosis is most extensive in early disease stages and decreases over time. Brain 125, 2202-2212
40. van Leeuwen, E. M., Remmerswaal, E. B., Vossen, M. T., Rowshani, A. T., Wertheim-van Dillen, P. M., van Lier, R. A., and ten Berge, I. J. (2004) Emergence of a CD4+ CD28− granzyme B+, cytomegalovirus-specific T cell subset after recovery of primary cytomegalovirus infection. J Immunol 173, 1834-1841
41. Martens, P. B., Goronzy, J. J., Schaid, D., and Weyand, C. M. (1997) Expansion of unusual CD4+ T cells in severe rheumatoid arthritis. Arthritis Rheum 40, 1106-1114
42. Trapani, J. A. (2001) Granzymes: a family of lymphocyte granule serine proteases. Genome Biol 2, REVIEWS3014
43. Kurschus, F. C., Bruno, R., Fellows, E., Falk, C. S., and Jenne, D. E. (2005) Membrane receptors are not required to deliver granzyme B during killer cell attack. Blood 105, 2049-2058
44, Tepe, N. M., and Liggett, S. B. (2000) Functional receptor coupling to Gi is a mechanism of agonist-promoted desensitization of the beta2-adrencrgic receptor. J Recept Signal Transduct Res 20, 75-85
45. Suidan, H. S., Bouvier, J., Schaerer, E., Stone, S. R., Monard, D., and Tschopp, J. (1994) Granzyme A released upon stimulation of cytotoxic T lymphocytes activates the thrombin receptor on neuronal cells and astrocytes. Proc Nail Acad Sci USA 91, 8112-8116
46. Dery, 0., Corvera, C. U., Steinhoff, M., and Bunnett, N. W. (1998) Proteinase-activated receptors: novel mechanisms of signaling by serine proteases. Am. J Physiol 274, C1429-1452
47. Vanhoose, A. M., Ritchie, M. D., and Winder, D. G. (2004) Regulation of cAMP levels in area CA1 of hippocampus by Gi/o-coupled receptors is stimulus dependent in mice. Neurosci Lett 370, 80-83
48. Abel, T., Nguyen, P. V., Barad, M., Deuel, T. A., Kandel, E. R., and Bourtchouladze, R. (1997) Genetic demonstration of a role for PKA in the late phase of LTP and in hippocampus-based long-term memory. Cell 88, 615-626
49. Wong, S. T., Athos, J., Figueroa, X. A., Pineda, V. V., Schaefer, M. L., Chavkin, C. C., Muglia, L. J., and Storm, D. R. (1999) Calcium-stimulated adenylyl cyclase activity is critical for hippocampus-dependent long-term memory and late phase LTP. Neuron 23, 787-798
50. Pashenkov, M., Soderstrom, M., and Link, H. (2003) Secondary lymphoid organ chemokines are elevated in the cerebrospinal fluid during central nervous system inflammation. J Neuroimmunol 135, 154-160
51. Lu, F., Selak, M., O'Connor, J., Croul, S., LorenZana, C., Butunoi, C., and Kalman, B. (2000) Oxidative damage to mitochondrial DNA and activity of mitochondrial enzymes in chronic active lesions of multiple sclerosis. J Neurol Sci 177, 95-103
52. Kalman, 13., and Leist, T. P. (2003) A mitochondrial component of neurodegeneration in multiple sclerosis. Neuromolecular Med 3, 147-158

1. A method of identifying test agents as neuroprotective agents comprising:
a. contacting a first sample of cells which express Kv1.3 with Granzyme B in the presence of a test agent;
b. contacting a second sample of said cells with Granzyme B in the absence of a test agent;
c. determining viability of the cells in (a) and (b) after the Contacting;
d. comparing determined viability of the cells from (c);
e. identifying the test agent as a candidate neuroprotective agent if the viability of the cells is higher in (a) than in (b).

2. The method of claim 1 further comprising assaying the test agent for binding to Kv1.3 channels.

3. The method of claim 1 further comprising contacting a third sample of said cells with Granzyme B in the presence of a test agent and Margotoxin.

4. The method of claim 1 wherein the cells express Kv1.3 from an exogenous expression construct.

5. The method of claim 1 wherein the cells are kidney cells.

6. The method of claim 1 wherein the cells are human cells.

7. The method of claim 1 wherein the cells are neuronal cells.

8. The method of claim 1 wherein the viability of the cells is determined by dye staining.

9. The method of claim 1 wherein the viability of the cell is determined by assessing apoptosis.

10. The method of claim 1 wherein the viability of the cell is determined by assessing chromatin condensation.

11. The method of claim 1 wherein the viability of the cell is determined by assessing caspase activation.

12. A method of treating a mammal with a neurodegenerative disease or neural injury, comprising:
administering a specific Kv1.3 channel inhibitor directly to the central nervous system (CNS) of the mammal, whereby loss of viable neurons is inhibited, and/or growth of neuronal processes is stimulated, and/or proliferation of neuronal precursor cells is stimulated.

13. A method of treating a mammal with a neural injury or a neurodegenerative disease not associated with pathological T cell activation, comprising:
administering a specific Kv 1.3 channel inhibitor to the mammal whereby loss of viable neurons is inhibited, and/or growth of neuronal processes is stimulated, and/or proliferation of neuronal precursor cells is stimulated.

14. The method of claim 12 or 13 wherein the specific inhibitor is SHK22DAP.

15. The method of claim 12 or 13 wherein the specific inhibitor is SL5.

16. The method of claim 12 or 13 wherein the specific inhibitor is PAP1.

17. The method of claim 12 or 13 wherein the specific inhibitor is khellinone.

18. The method of claim 12 or 13 wherein the specific inhibitor is 8-methoxypsoralen.

19. The method of claim 12 or 13 wherein the specific inhibitor is 5-methoxy psoralen.

20. The method of claim 12 wherein the Mammal is a human with a neurodegenerative disease, and the disease is multiple sclerosis.

21. The method of claim 12 wherein the mammal has sustained a traumatic neural injury.

22. The method of claim 12 wherein the mammal is a human with a neurodegenerative disease, and the disease is multiple sclerosis.

23. The method of claim 12 or 13 wherein the mammal has sustained a traumatic neural injury.

24. The method of claim 12 or 13 wherein the disease is Parkinson's Disease.

25. The method of claim 12 or 13 wherein the disease is Alzheimer's Disease.

26. The method of claim 12 or 13 wherein the mammal has Parkinsonism.

27. The method of claim 12 or 13 wherein the mammal has dementia.

28. The method of claim 12 or 13 wherein the specific inhibitor is an antibody which binds to Kv1.3 channels.

29. A method of treating a mammal with a neurodegenerative disease or neural injury, comprising:
administering a specific inhibitor of Kv 1.3 channel expression directly to the central nervous system (CNS) of the mammal, whereby loss of viable neurons is inhibited, and/or growth of neuronal processes is stimulated, and/or proliferation of neuronal precursor cells is stimulated.

30. A method of treating a mammal with a neural injury or a neurodegenerative disease not associated with pathological T cell activation, comprising:
administering a specific inhibitor of Kv 1.3 channel expression to the mammal thereby loss of viable neurons is inhibited, and/or growth of neuronal processes is stimulated, and/or proliferation of neuronal precursor cells is stimulated.

31. The method of claim 29 or 30 wherein the specific inhibitor is a nucleic acid molecule.

32. The method of claim 29 or 30 wherein the specific inhibitor is a siRNA molecule.

33. The method of claim 29 or 30 wherein the specific inhibitor is an antisense RNA molecule.

34. The method of claim 29 or 30 wherein the specific inhibitor is an antisense construct from which antisense RNA is expressed.
